(19) United States
(12) Patent Application Publication
(10) Pub. No.: US 2026/0144938 A1
(43) Pub. Date: May 28, 2026

(54) DRUG DELIVERY DEVICES AND METHODS FOR PRECISION DELIVERY OF INJECTABLE DRUGS

(71) Applicant: Congruence Medical Solutions, LLC, Baltimore, MD (US)

(72) Inventors: Lou CASTAGNA, Middletown, PA (US); Andrew SCHAEFER, Monkton, MD (US); Gautam Nithyanand SHETTY, Hanover, MD (US)

(73) Assignee: Congruence Medical Solutions, LLC, Baltimore, MD (US)

(21) Appl. No.: 19/121,765

(22) PCT Filed: Oct. 17, 2023
(86) PCT No.: PCT/US2023/077122

§ 371 Date: Apr. 16, 2025

Related U.S. Application Data
(60) Provisional application No. 63/416,825, filed on Oct. 17, 2022.

(51) Int. Cl. A61M 5/315; A61M 5/31

(52) U.S. Cl. CPC A61M 5/31501; A61M 5/31515; A61M 5/3153; A61M 5/31536; A61M 5/3157; A61M 5/31578; A61M 5/31591; A61M 2005/3126; A61M 2005/31508; A61M 2005/3154; A61M 2202/0007; A61M 2202/04; A61M 2205/27; A61M 2205/581; A61M 2205/582; A61M 2205/583

(57) ABSTRACT

Described herein are methods and systems for operating a syringe-based drug delivery device to eject a dose of a fluid from a delivery conduit, such as a needle. The methods involve advancing a plunger rod of the syringe-based drug delivery device in an axial direction of the plunger rod from a start of dose position to an end of dose position. This advances a plunger stopper within a barrel of the syringe-based drug delivery device. The plunger stopper is compressed when the plunger rod first reaches the end of dose position. The difference between the start of dose position and the end of dose position accounts for compression of the plunger stopper caused by resistance of the fluid to ejection through the delivery conduit.

### CROSS-REFERENCE TO RELATED APPLICATIONS

This application claims priority to U.S. Provisional Patent Application No. 63/416,825, entitled “Novel Drug Delivery Device for Precision Delivery of Injectable Drugs,” filed Oct. 17, 2022, the disclosure of which is incorporated herein by reference in its entirety.

### TECHNICAL FIELD

This application relates generally to injectable drug delivery devices, and, more particularly, to devices for the injection of sub-milliliter volumes of viscous injectable formulations.

### BACKGROUND

Syringes are commonly used for administration of injectable therapeutics or fluids. As shown in FIG. 1, syringes typically consist of a cylindrical Syringe Barrel, an elastomeric plunger stopper that glides along of the axis of the Syringe Barrel upon user manipulation of a plunger rod, and a Needle. The plunger stopper, Syringe Barrel, and the Needle encapsulate the drug fluid chamber, with the needle orifice providing the only outlet for the drug fluid.

Increasingly, the viscosity of drug formulations is increasing due to the advent of high-strength biologic drugs, bi-specific antibody therapeutics, and long-acting formulations. The Hagen-Poiseuille equation (reproduced below) can be used to demonstrate the relationship between the injection force (F) required during drug delivery and the viscosity (μ) of the formulation.

$$F = \frac{128 Q μ LA}{π {D}^{4}}$$

$$Q - volumetric flow rate , μ - dynamic viscosity , L - needle length , D - needle internal diameter , A - s yringe internal cross section area$$

The Hagen-Poiseuille equation shows how increasing viscosity leads to a higher injection force required, as well as how other parameters impact injection force such as needle internal diameter and syringe diameter. The Hagen-Poiseuille equation also indicates that a very fine needle, such as those used in subretinal injections, can cause the injection force to be high even if the viscosity of the injected drug formulation is low.

In a conventional syringe, the elastomeric plunger stopper is compressible. When the user advances the plunger rod to administer the drug contained in the syringe, the drug solution is pressurized, which drives flow through the needle. For highly viscous formulations and/or when the formulation is injected through a fine gauge needle, the pressure may increase within the fluid chamber to such an extent that it causes compression of the plunger stopper even as the drug is being dispensed through the needle. Therefore, some part of the user input to inject the drug fluid may result in axial compression (along with radial displacement) of the plunger stopper rather than injection of the drug fluid. In this case, even if the user's tactile feedback may suggest an injection is occurring, in reality the user may only be compressing the plunger. In case of larger volumes (milliliters), this effect (axial compression of the plunger stopper) is a small proportion of the axial travel of the plunger stopper to administer the entire dose. However, for sub-milliliter volumes, the extent of axial compression of the plunger stopper, for highly viscous formulations and/or when the formulation is injected through a fine gauge needle, may be a significant relative to the plunger stopper axial travel to deliver the intended dose volume. Therefore, if the amount of axial user input does not account for the axial compression, the plunger stopper may not achieve the axial travel necessary to administer the entire dose, and an underdose may be the result.

In applications for which the user (e.g., a clinician) is not able to visually inspect the plunger stopper during the injection, such as injections into the eye, the user may rely entirely on tactile feel, which, as described in the aforementioned situation with sub-milliliter doses, can be misleading. Also, injection devices that incorporate conventional syringes are designed to axially advance the plunger stopper by a distance without factoring in compression of the plunger stopper, which may cause a significant underdose when administering sub-milliliter volume of drugs. Therefore, viscous formulations, sub-milliliter dose volumes, and/or injecting a sub-milliliter volume through a very fine injection needle may amplify drug delivery challenges with a syringe or syringe-based system.

### SUMMARY

Embodiments of the present disclosure are directed toward methods and syringe-based drug delivery device designs to address challenges in applications involving high injection forces to inject sub-milliliter volumes. In certain embodiments, an exemplary injection device may ensure that an accurate and precise sub-milliliter dose is injected when plunger stopper compression occurs. In order to account for plunger stopper compression, the injection stroke provided by the user may be the sum of theoretical axial plunger stopper travel distance to dispense the anticipated dose and the axial plunger stopper compression anticipated for the injection force. For example,

$$Injection Stroke = Theoretical Axial plunger stopper travel for target dose + Anticipated$$

### Axial Plunger Stopper Compression

In the case of this exemplary manual injection device, if the total plunger stopper travel to inject a dose is two millimeters, but there is one millimeter of axial plunger stopper compression corresponding to the expected injection force, then the injection stroke to deliver the target dose may be three (two+one) millimeters. If there is room for axial decompression of the stopper, the injection needle may be removed following the completion of the injection stroke before decompression occurs. In some embodiments, the dose may be a drug that is prefilled in the syringe and/or transferred from a vial into the syringe.

The exemplary injection device may include a gear train involving a compound gear that is configured to attenuate the injection force experienced by the user. Accordingly, the user input force may be amplified, and a smaller user input force may generate a higher injection force to advance the plunger stopper. Such a device can also provide the user with stability in case of sensitive applications such as injections in the eye. Some embodiments may not employ a gear train.

Unlike conventional syringes, the exemplary injection device described herein may have an injection stroke that ends when the plunger rod contacts the housing of the exemplary injection device. Advancing the plunger stopper to the start of dose position may be achieved via a ratcheted mechanism configured to prevent the plunger rod from being kicked back opposite to the user input as it is being axially advanced during the priming step. Furthermore, unlike conventional syringes, an exemplary injection device may include a plunger rod locking mechanism configured to maintain the plunger rod's axial position at the end of the dose. This feature may ensure that the only outlet/relief for the pressurized drug chamber is for the drug to exit through the needle.

According to some embodiments, described herein is an exemplary method comprising: advancing a plunger rod of a syringe-based drug delivery device in an axial direction of the plunger rod from a start of dose position to an end of dose position to advance a plunger stopper within a barrel of the syringe-based drug delivery device for ejecting a dose of a fluid from a delivery conduit of the syringe-based drug delivery device, wherein the plunger stopper is compressed when the plunger rod first reaches the end of dose position, and a difference between the start of dose position and the end of dose position accounts for compression of the plunger stopper caused by resistance of the fluid to ejection through the delivery conduit.

According to some embodiments, the end of dose position is defined by abutment between a portion of the plunger rod and a body of the syringe-based drug delivery device.

According to some embodiments, the end of dose position is defined by at least one of a visual marker, an audible indicator, and a tactile indicator.

According to some embodiments, the plunger stopper is mounted at one end to an end of the plunger rod such that the end of the plunger stopper that is mounted to the end of the plunger rod travels the difference between the start of dose position and the end of dose position.

According to some embodiments, the plunger stopper is coupled to a drive rod that is at least partially disposed within the barrel of the syringe-based drug delivery device, and the drive rod and the plunger rod are operatively coupled such that the drive rod and the plunger rod translate different amounts.

According to some embodiments, the plunger rod is axially locked at the end of dose position.

According to some embodiments, prior to advancing the plunger rod of the syringe-based drug delivery device from the start of dose position to the end of dose position, the exemplary method may further comprise advancing the plunger rod to the start of dose position, and waiting for the fluid to stop exiting the delivery conduit. In some embodiments, the exemplary method may further comprise visually, audibly, and/or tactilely indicating the start of dose position of the plunger rod.

According to some embodiments, the plunger rod engages a ratchet configured to prevent the plunger rod from being pushed backward due to the compression of the plunger stopper.

According to some embodiments, prior to advancing the plunger rod of the syringe-based drug delivery device from the start of dose position to the end of dose position, the exemplary method may further comprise inserting the delivery conduit into an injection site, and withdrawing the delivery conduit from the injection site prior to decompression of the plunger stopper.

According to some embodiments, a difference between a start of dose position of a fluid contacting end of the plunger stopper and an end of dose position of the fluid contacting end of the plunger stopper is less than the difference between the start of dose position and the end of dose position of the plunger rod when the delivery conduit is withdrawn from the injection site.

According to some embodiments, described herein is an exemplary syringe-based drug delivery device comprising: a housing comprising a barrel for containing a dose of a fluid; a delivery conduit coupled to the barrel and configured for insertion into an injection site for delivering the dose of the fluid; a plunger stopper positioned in the barrel; a plunger rod operatively coupled to the plunger stopper to move the plunger stopper to deliver the dose of the fluid; and at least one of a visual, audible, and tactile indicator of each of: (a) a start of dose position of the plunger rod and (b) an end of dose position of the plunger rod, wherein a difference between the start of dose position and the end of dose position accounts for compression of the plunger stopper during delivery of the dose of the fluid.

According to some embodiments, at least one of a visual, audible, and tactile indicator of the end dose position comprises an abutment between the plunger rod and a housing of the syringe-based drug delivery device that prevents the plunger rod from being further advanced.

According to some embodiments, the plunger stopper is mounted at one end to an end of the plunger rod such that the end of the plunger stopper that is mounted to the end of the plunger rod travels the difference between the start of dose position and the end of dose position.

According to some embodiments, the plunger stopper is mounted to a drive rod that is at least partially disposed within the barrel of the syringe-based drug delivery device, and the drive rod and plunger rod are operatively coupled such that the drive rod and the plunger rod translate different amounts.

According to some embodiments, the plunger rod engages the housing at the end of dose position so that the plunger rod cannot move backward from the end of dose position.

According to some embodiments, the exemplary device may further comprise a ratchet configured to prevent the plunger rod from being pushed backward due to decompression of the stopper as the plunger is being advanced to the start of dose position.

According to some embodiments, a difference between a start of dose position of a fluid contacting end of the plunger stopper and an end of dose position of the fluid contacting end of the plunger stopper is less than the difference between the start of dose position and the end of dose position of the plunger rod when the delivery conduit is withdrawn from the injection site.

### BRIEF DESCRIPTION OF THE DRAWINGS

The invention will now be described, by way of example only, with reference to the accompanying drawings, in which:

FIG. 1 illustrates an external view of a conventional syringe.

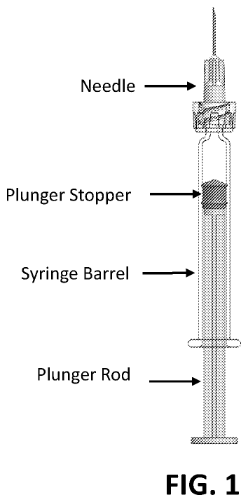

FIGS. 2A-2D illustrate external views of various stages of operation of a conventional syringe.

FIG. 3 illustrates an exploded view of an exemplary syringe-based drug delivery device that includes a mechanism to attenuate the injection force experienced by the user.

FIGS. 4A-4E illustrate external views of various stages of operation of an exemplary syringe-based drug delivery device that includes a mechanism to attenuate the injection force experienced by the user.

FIGS. 5A-5E illustrate cross-sectional views of various stages of operation of an exemplary syringe-based drug delivery device having a plunger rod. The plunger rod is configured to be locked into an end of dose position via a locking position.

FIG. 6 illustrates an external view of a mechanism to attenuate the injection force experienced by the user. The mechanism is part of an exemplary syringe-based drug delivery device.

FIGS. 7A-7D illustrate cross-sectional views of an interaction between a dial and a drive rod of an exemplary syringe-based drug delivery device.

FIGS. 8A-8C illustrate cross-sectional views of a ratcheting mechanism to advance a plunger rod and a drive rod towards a start of dose position. The ratcheting mechanism is part of an exemplary syringe-based drug delivery device.

FIGS. 9A-9E illustrates various views of a dial and a ratcheting mechanism configured to be combined into one part. The dial and ratcheting mechanism are part of an exemplary syringe-based drug delivery device.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein, according to various embodiments, are syringe-based drug delivery devices that include mechanisms for preventing underdosing during the injection of sub-milliliter volumes of viscous injectable formulations. An exemplary syringe-based drug delivery device can include a visual, audible, and/or tactile indicator for indicating when a full injection stroke has been completed. The indicators can account for the compression of the plunger stopper during the injection of a formulation, thereby providing an accurate dose instead of an underdose.

The exemplary device can include a barrel containing a dose of the formulation (i.e., an injectable drug). One end of the barrel may lead to a needle through which the formulation can be ejected. Another end of the barrel may lead to a plunger stopper for pushing the formulation out of the needle. The compression of the plunger stopper may be caused by the resistance of the formulation to ejection through the needle. The plunger stopper may be operatively coupled to a plunger rod, which can be pushed by a user to deliver the dose of the formulation. Because the plunger stopper may be compressible by nature, pushing the plunger rod by a distance “X” may not mean that the plunger stopper moves the entire distance “X.” Instead, part of the plunger stopper may be compressed in size, which means that not all of the formulation may be pushed out of the barrel through the needle (i.e., underdosing). To deliver an accurate dose of the formulation, the plunger rod may be advanced in an axial direction through a full injection stroke, beginning from a “start of dose” position and ending at an “end of dose” position. This, in turn, advances the plunger stopper through the barrel, pushing the formulation out of the needle. The plunger stopper may be compressed when the plunger rod first reaches the “end of dose” position.

The locations of the “start of dose” and “end of dose” positions can be set to account for the compression of the plunger stopper, allowing for the full dose of formulation to be dispensed. As described above, the indicators may alert the user when the “start of dose” and “end of dose” positions are reached, allowing the user to accurately determine that the full injection stroke has been completed. In some embodiments, the device may include a numbered dial that rotates when the plunger rod is pressed. The change in the numbers of the dial may correspond to the specific positions of the dose.

A method for using a syringe-based drug delivery device can include advancing a plunger rod of a syringe-based drug delivery device in an axial direction of the plunger rod from a start of dose position to an end of dose position to advance a plunger stopper within a barrel of the syringe-based drug delivery device for ejecting a dose of a fluid from a delivery conduit of the syringe-based drug delivery device, wherein the plunger stopper is compressed when the plunger rod first reaches the end of dose position, and a difference between the start of dose position and the end of dose position accounts for compression of the plunger stopper caused by resistance of the fluid to ejection through the delivery conduit. The end of dose position may be defined by abutment between a portion of the plunger rod and a body of the syringe-based drug delivery device. The end of dose position may be defined by at least one of a visual marker, an audible indicator, and a tactile indicator. The plunger stopper may be mounted at one end to an end of the plunger rod such that the end of the plunger stopper that is mounted to the end of the plunger rod travels the difference between the start of dose position and the end of dose position. The plunger stopper may be coupled to a drive rod that is at least partially disposed within the barrel of the syringe-based drug delivery device, and the drive rod and the plunger rod may be operatively coupled such that the drive rod and the plunger rod translate different amounts. The plunger rod may be axially locked at the end of dose position.

Prior to advancing the plunger rod of the syringe-based drug delivery device from the start of dose position to the end of dose position, the exemplary method may further include advancing the plunger rod to the start of dose position, and waiting for the fluid to stop exiting the delivery conduit. In some embodiments, the exemplary method may further comprise visually, audibly, and/or tactilely indicating the start of dose position of the plunger rod. The plunger rod may engage a ratchet configured to prevent the plunger rod from being pushed backward due to the compression of the plunger stopper. Optionally, prior to advancing the plunger rod of the syringe-based drug delivery device from the start of dose position to the end of dose position, the exemplary method may further comprise inserting the delivery conduit into an injection site, and withdrawing the delivery conduit from the injection site prior to decompression of the plunger stopper. A difference between a start of dose position of a fluid contacting end of the plunger stopper and an end of dose position of the fluid contacting end of the plunger stopper may be less than the difference between the start of dose position and the end of dose position of the plunger rod when the delivery conduit is withdrawn from the injection site.

According to an aspects, an exemplary syringe-based drug delivery device may include a housing comprising a barrel for containing a dose of a fluid; a delivery conduit coupled to the barrel and configured for insertion into an injection site for delivering the dose of the fluid; a plunger stopper positioned in the barrel; a plunger rod operatively coupled to the plunger stopper to move the plunger stopper to deliver the dose of the fluid; and at least one of a visual, audible, and tactile indicator of each of: (a) a start of dose position of the plunger rod and (b) an end of dose position of the plunger rod, wherein a difference between the start of dose position and the end of dose position accounts for compression of the plunger stopper during delivery of the dose of the fluid. The at least one of a visual, audible, and tactile indicator of the end dose position may include an abutment between the plunger rod and a housing of the syringe-based drug delivery device that prevents the plunger rod from being further advanced.

The plunger stopper may be mounted at one end to an end of the plunger rod such that the end of the plunger stopper that is mounted to the end of the plunger rod travels the difference between the start of dose position and the end of dose position. The plunger stopper may be mounted to a drive rod that is at least partially disposed within the barrel of the syringe-based drug delivery device, and the drive rod and plunger rod are operatively coupled such that the drive rod and the plunger rod translate different amounts. The plunger rod may engage the housing at the end of dose position so that the plunger rod cannot move backward from the end of dose position. The exemplary device may further include a ratchet configured to prevent the plunger rod from being pushed backward due to decompression of the stopper as the plunger is being advanced to the start of dose position. A difference between a start of dose position of a fluid contacting end of the plunger stopper and an end of dose position of the fluid contacting end of the plunger stopper may be less than the difference between the start of dose position and the end of dose position of the plunger rod when the delivery conduit is withdrawn from the injection site

In the following description of the various embodiments, reference is made to the accompanying drawings, in which are shown, by way of illustration, specific embodiments that can be practiced. It is to be understood that other embodiments and examples can be practiced and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain terminology is used in this description for convenience and reference only and is not limiting. For example, the words “upwardly,” “downwardly,” “rightwardly,” and “leftwardly” refer to directions in the drawings to which reference is made. The words “inwardly” and “outwardly” refer to directions toward and away from, respectively, the geometric center of the arrangement and designated parts thereof. The words “forwardly” and “distally” refer to the direction toward the end of the arrangement that is closest to the subject, and the words “rearwardly” and “proximally” refer to the direction toward the end of the arrangement which is furthest from the subject. This terminology includes the words specifically mentioned, derivatives thereof, and words of a similar nature.

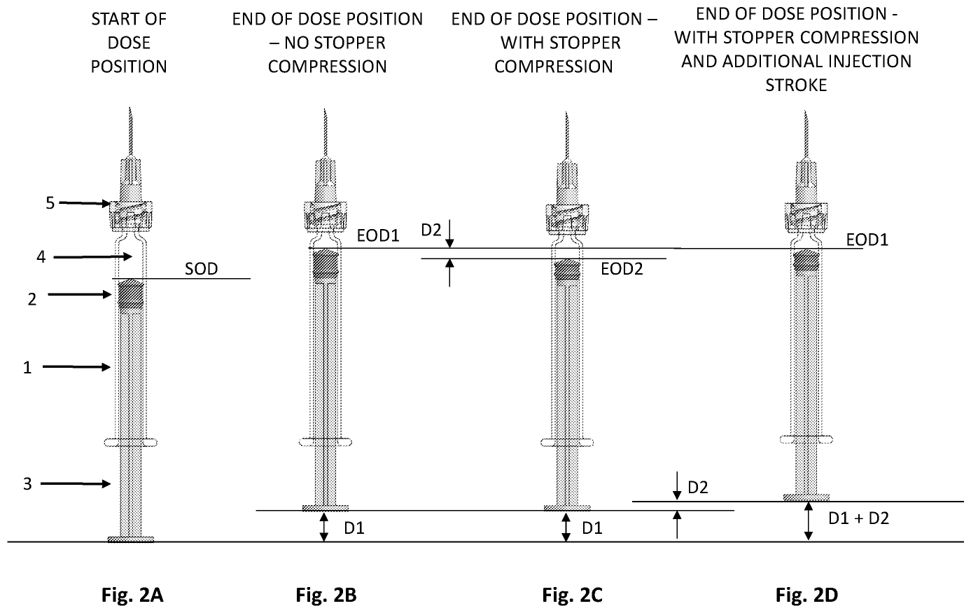

FIGS. 2A-2D illustrate various stages of operation of a syringe-based delivery system that includes of a cylindrical syringe barrel 1. The syringe barrel 1 may or may not be lubricated on its internal surface to enable an elastomeric plunger stopper 2 to axially translate along a central axis of the syringe barrel 1 using a plunger rod 3, which is configured to be manipulated by the user. Typically, the user adjusts the location of the plunger stopper 2 to the start of dose position by aligning a part (e.g., the tip) of the plunger stopper 2 with dose volume markings (not shown) printed on the outer surface of syringe barrel 1. In the illustrated embodiment, the start of dose position is visually indicated by a marker 12 on the plunger rod 3 that, when aligned with a proximal end of the syringe barrel 1, indicates that the plunger stopper 2 is in the start of dose position. In some embodiments, a tactile and/or audible indicator of the start of dose position may be provided. For example, a protrusion on the plunger rod 3 may contact a portion of the syringe barrel 1 providing tactile feedback to the user and/or an audible sound. An injectable drug 4 may be contained within the syringe barrel 1. Once at the start of dose position, the user may wait until the injectable drug 4 stops exiting the needle of the needle assembly 5, which may indicate that the plunger stopper 2 has decompressed. For injections with a delivery conduit (e.g., needle assembly 5, including a needle), the user may insert the needle of the needle assembly 5 into the target injection site and axially depresses the plunger rod 3 until it reaches the end of dose position. The end of dose position may be defined by abutment between a portion of the plunger rod 3 and syringe barrel 1 and/or may be visually, audibly, and/or tactilely indicated to the user. In the illustrated embodiment, an end of dose position indicator 12 is included on the plunger rod 3. Alignment between this indicator 12 and a proximal end of the syringe barrel 1 may indicate the start of dose position.

In cases where there is no compression of the plunger stopper 2, the end of dose position may be when the plunger stopper reaches the bottom of the syringe barrel 1. Distance D1 is the length of the injection stroke along the axial direction of the plunger rod 3 as it goes from start of dose position SOD to end of dose position EOD1 to dispense a sub-milliliter injection volume V.

FIG. 2C illustrates a syringe-based delivery system in a scenario where plunger stopper 2 is compressed by a distance D2, such that the end of dose position is now EOD2. The difference in end of dose positions EOD2 and EOD1 (equal to D2) would correspond to the volume of drug not dispensed (i.e., underdose) due to the compression of the plunger stopper 2 if the plunger rod 3 were not advanced further and the needle of the needle assembly 5 were withdrawn from the injection site prior to stopper decompression. In order to dispense the same volume of drug V corresponding to the scenario in FIG. 2B (and also have plunger stopper 2 reach the same end of dose position EOD1), an additional injection stroke D2 is required as illustrated in FIG. 2D. Distance D2 is the difference between EOD1 and EOD2 along the axial direction of the plunger rod 3. Therefore, when plunger stopper 2 compression occurs when injecting a sub-milliliter volume, the injection stroke to deliver sub-milliliter injection volume V would be D1+D2, which is illustrated in FIG. 2D. Thus, the syringe-based delivery system can include a visual, audible, or tactile indicator of the end of dose position of the plunger rod 3. In the illustrated embodiments, this is in the form of a visual indicator 12 on the plunger rod 3. The user may push the plunger rod 3 until the visual indicator 12 aligns with a proximal end of the syringe barrel 1. Once this occurs, the full dose has been dispensed and the user can withdraw the needle from the injection site before decompression of the plunger stopper 2 occurs.

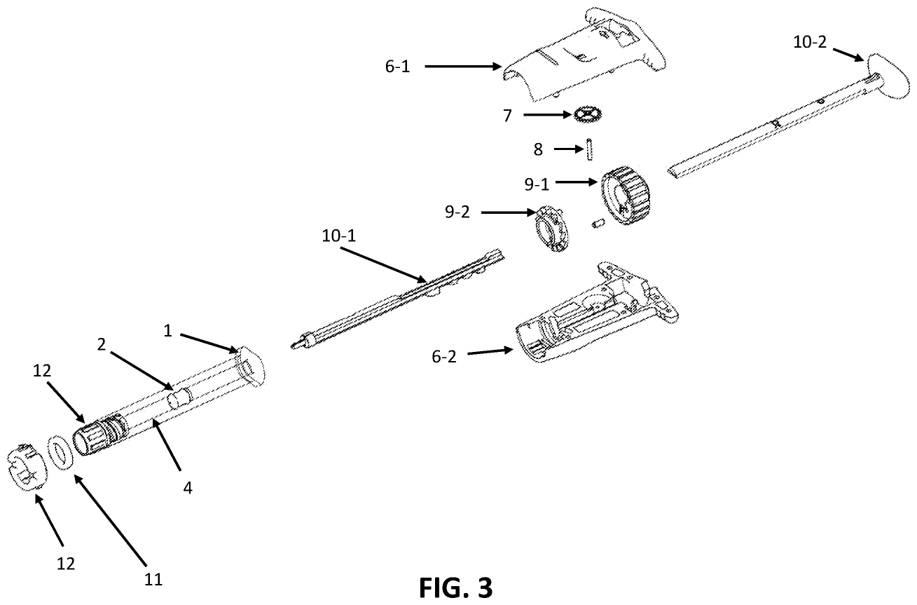

FIG. 3 illustrates an exploded view of an exemplary syringe-based drug delivery device that includes a mechanism to attenuate the injection force experienced by the user. The device may include a cover 6-1 and housing 6-2, both of which collectively may be regarded as the body of the device. Cover 6-1 and housing 6-2 may include mating features configured to securely attach to each other. The device may comprise a plunger rod 10-2 and a drive rod 10-1, which interact with compound gear 7, which may be axially constrained inside the body of the device by pin 8. The device may comprise a rotational ratchet 9-2 along with a beam (not shown) on housing 6-2, which may be configured to ensure that plunger rod 10-2 and drive rod 10-1 do not experience retrograde travel from any decompression forces exerted by the plunger stopper 2 as they are advanced to the start of dose position. The device may comprise a dial 9-1, which may interact with features on the drive rod 10-1 to axially advance the drive rod 10-1 (by way of compound gear 7 and/or plunger rod 10-2) towards the start of dose position. In some embodiments, the dial 9-1 and rotational ratchet 9-2 may be combined as one part. In some embodiments, a drug 4 may be prefilled in a syringe barrel 1 of the device and enclosed by plunger stopper 2 and tip cap 12. Syringe barrel 1 may be secured to the body (e.g., cover 6-1 and housing 6-2) of the device using clip 12. An elastomeric, circular o-ring (or x-ring) may be placed within the device body to minimize rotational slip of the syringe barrel 1 relative to the device body.

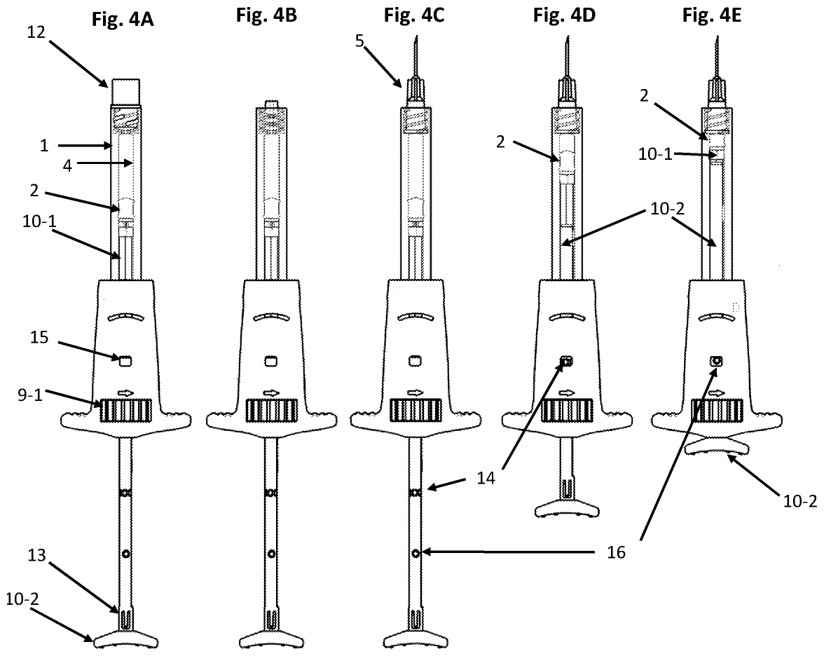

FIGS. 4A-4E show external views of various stages of operation of an exemplary syringe-based drug delivery device, such as the device of FIG. 3. FIG. 4A illustrates the device as received by the user once prefilled with drug 4. FIG. 4B illustrates the removal of the tip cap 12 from the syringe barrel 1. FIG. 4C illustrates the attachment of a needle assembly 5 to the syringe barrel 1 in place of the tip cap 12. FIG. 4D illustrates the advancement of the plunger stopper 2 to the start of dose position. Turning the dial 9-1 advances the plunger rod 10-2 and drive rod 10-1, which in turn advances plunger stopper 2 towards the start of dose position. In some embodiments, the drive rod 10-1 may be at least partially disposed within the syringe barrel 1. The drive rod 10-1 and the plunger rod 10-2 may be operatively coupled such that the drive rod 10-1 and the plunger rod 10-2 translate by different amounts when the dial 9-1 is rotated. In some embodiments, one end of the plunger stopper 2 may be mounted to an end of the plunger rod 10-2 such that the one end of the plunger stopper 2 travels at least a portion of the distance between the start of dose position and the end of dose position. In some embodiments, the start of dose position and/or the end of dose position may be defined by at least one of a visual marker, an audible indicator, and/or a tactile indicator. In some embodiments, the plunger rod 10-2 may be axially locked at the end of dose position.

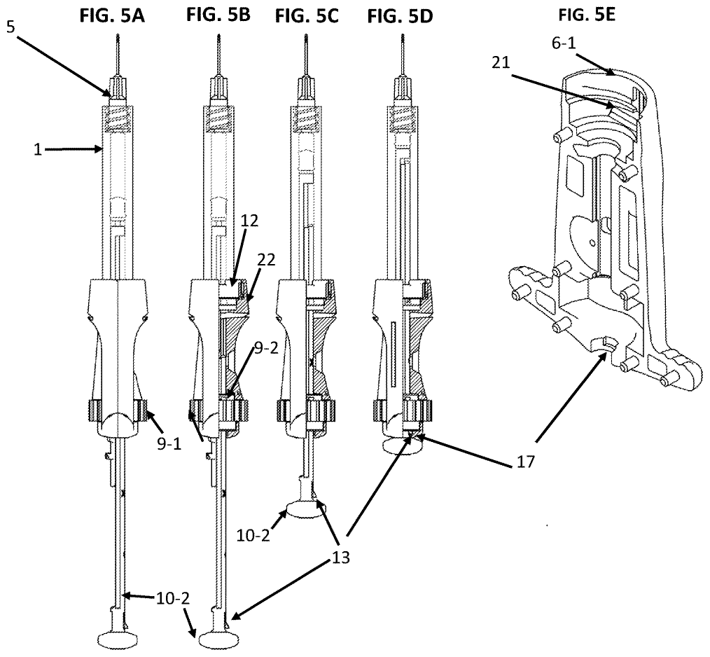

At the start of dose position shown in FIG. 4D, the printed dose amount 14 may be visible in window 15, providing visual confirmation. Concurrently, the dial 9-1 may reach a hard stop (not shown here) at the start of dose position and may not turn. In some embodiments, prior to advancing the plunger rod 10-2 from the start of dose position to the end of dose position, the plunger rod 2 may be kept in the start of dose position until fluid (e.g., from the drug 4) stops exiting the needle assembly 5. For example, once the start of dose position is reached, the ratchet 9-2 may prevent retrograde travel of the drive rod 10-1 as the plunger stopper 2 decompresses. The user may wait until the drug 4 stops dripping out of the needle assembly 5 once the plunger stopper 2 has sufficiently decompressed. FIG. 4E illustrates the device after the end of dose position is reached. As shown, the plunger rod 10-2 may bottom out against the housing 6-2 of the device. Immediately at the end of dose position, the plunger stopper 2 may still be compressed. The user may remove the needle assembly 5 from the injection site after the plunger rod 10-2 contacts the body of the device (e.g., cover 6-1 and housing 6-2). In some embodiments, a locking feature 13 may lock into the cover 6-1 (as shown in FIG. 5E) of the device, which may prevent retrograde travel of plunger rod 10-2 and drive rod 10-1. Referring back to FIGS. 4A and 4E, the window 15 may show a printed mark 16 for visually confirming the end of dose. The travel of the plunger rod 10-2 from its position shown in FIG. 4D to the position shown for plunger rod 10-2 in FIG. 4E is the injection stroke; this travel may factor in the compression of the plunger stopper 2. The injection stroke may be the nominal travel for plunger stopper 2 to deliver the intended dose of drug 4 plus the estimated compression of the plunger stopper 2 given the expected injection force.

In some embodiments, prior to advancing the plunger rod 10-2 from the start of dose position to the end of dose position, the needle assembly 5 may be inserted into an injection site (e.g., an arm of a human patient). The plunger rod 10-2 may engage a ratcheting mechanism, such as ratchet 9-2 of FIGS. 5A-5D, to prevent the plunger rod from being pushed backward in response to the compression of the plunger stopper 2 (i.e., decompressing). The needle assembly 5 may be withdrawn from the injection site prior to the decompression of the plunger stopper 2. In some embodiments, a difference between a start of dose position and an end of dose position of the end of the plunger stopper 2 closer to the drug 4 may be less than the difference between the start of dose position and the end of dose position of the plunger rod 10-2 when the needle assembly 5 is withdrawn from the delivery site.

FIGS. 5A-5E illustrate the locking of plunger rod 10-2 of an exemplary syringe-based drug delivery device, such as the device of FIG. 3, at the end of dose position. FIG. 5A shows the device after needle assembly 5 is attached. FIG. 5B illustrates a cross-sectional view of the device, which exposes clip 12 of the device. The clip 12 may have two protrusions 22 (only one shown), which travels in track 21 of the cover 6-1 and also in housing 6-2 (not shown) in order to secure syringe 1 to the cover 601 and housing 6-2 (i.e., the body of the device). Ratchet 9-2 may be constrained by the body of the device and attached (e.g., rotationally and axially locked) with dial 9-1. FIG. 5C illustrates a device having a locking feature 13 along the plunger rod 10-2. As shown in FIG. 5C, the plunger rod 10-2 is located near the end of dose position but has not reached the position yet. At this position, a ramp feature of the locking feature 13 may be briefly deflected by its entry point in cover 6-1 towards the axis of the plunger rod 10-2. FIG. 5D illustrates the device of FIG. 5C after it has reached the end of dose position. As shown in FIG. 5D, the ramp feature of the locking feature 13, which may be proximal to the user, has been pushed past a step 17 on the cover 6-1. The locking feature 13 may no longer be deflected by cover 6-1, and hence, it may relax towards its home position (i.e., impacts a surface on cover 6-1), generating an audible indication that the end of dose position has been reached. The ramp feature of the locking feature 13 may be restricted by step 17 on the cover 6-1, which may prevent retrograde travel of the plunger rod 10-2. FIG. 5E illustrates the step 17, clip 21, and cover 6-1 in greater detail.

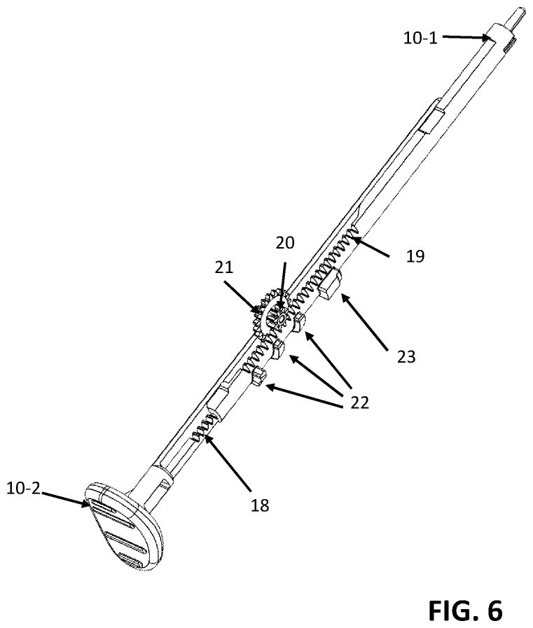

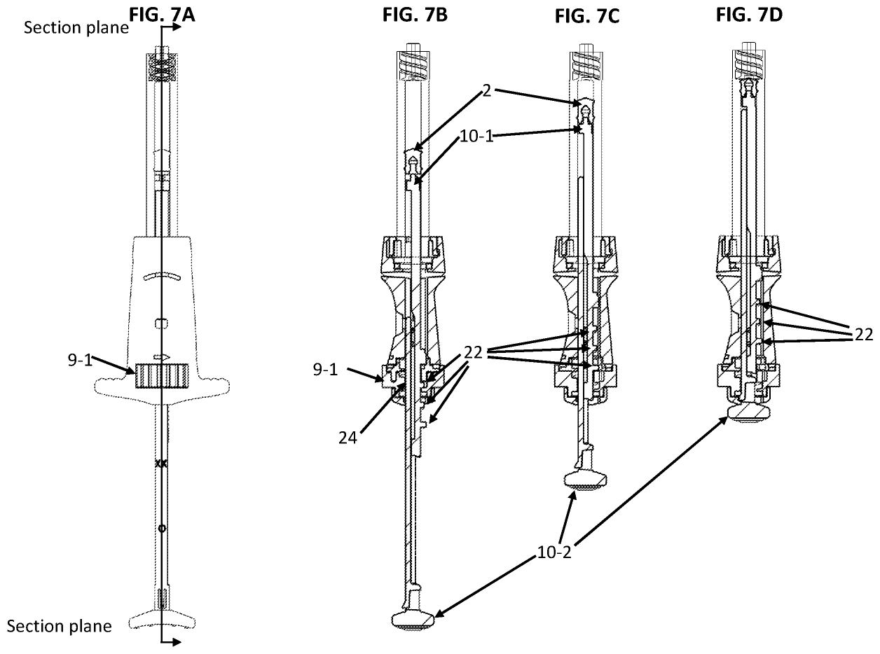

FIG. 6 illustrates a mechanism to attenuate the injection force experienced by the user when operating an exemplary syringe-based drug delivery device, such as the device of FIG. 3. The user may contact and/or manipulate the plunger rod 10-2. The plunger rod 10-2 may have sawtooth patterned teeth 18, which may mesh with the larger gear 21 of compound gear 7. Drive rod 10-1 may also include a row of teeth (rack) 19, which may mesh with smaller gear 20 of compound gear 7. In some embodiments, larger gear 21 and smaller gear 20 may together constitute compound gear 7. The ratio of the travel of plunger rod 10-2 to the travel of drive rod 10-1 may be equivalent to the ratio of the diameters of larger gear 21 and smaller gear 20. The ratio of the force experienced by the user in reaction to the injection force may be approximately inverse to the above ratio. Frictional forces involved in the moving parts may add to the force to be experienced by the user. In some embodiments, the attenuation mechanism may include a axial keying feature 23 configured to prevent rotation of the drive rod 10-1. The protrusions 22 of the drive rod 10-1 may interact with internal threads 24 in of dial 9-1 (as shown in FIGS. 7A-7D).

FIGS. 7A-7D show the interaction of the dial 9-1 with drive rod 10-1 of an exemplary syringe-based drug delivery device, such as the device of FIG. 3. FIG. 7A illustrates a sectional plane of the device. FIGS. 7B, 7C, and 7D illustrate cross-sectional views that show the states of the device once a needle (not illustrated) is attached, once the plunger rod 10-2 reaches the start of dose position, and once the plunger rod 10-2 reaches the end of dose position, respectively. Protrusions 22 of the drive rod 10-1 may interact with internal threads of dial 9-1 (as shown in FIG. 6). As the dial 9-1 is turned in one direction (e.g., a dose setting direction), the protrusions 22 may be axially advanced by the internal threads 24 until the protrusion of the protrusions 22 closest to the user exits the threads 24, and the start of dose position is reached, as shown in FIG. 7C. FIG. 7D illustrates the position of the protrusions when the end of dose position is reached.

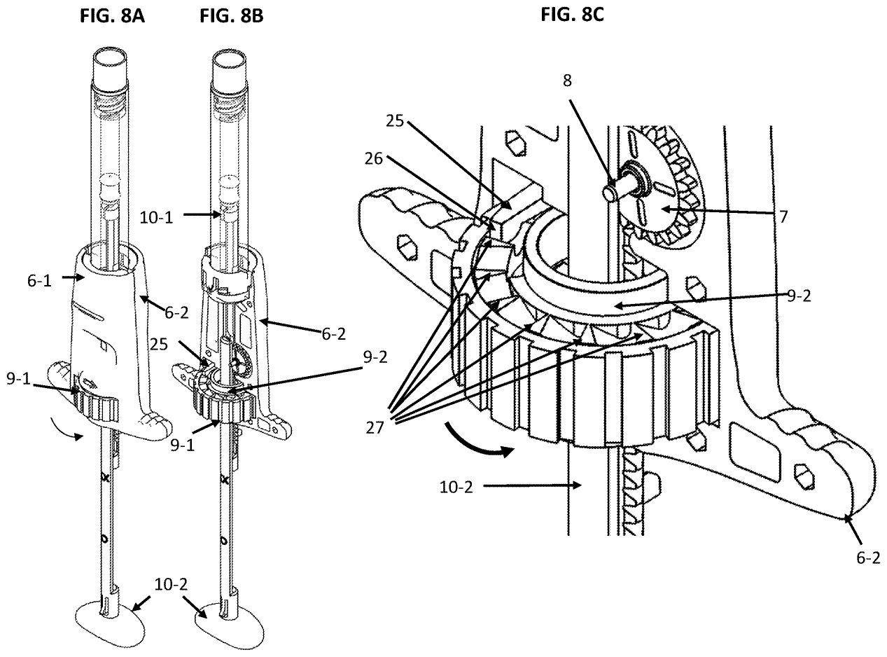

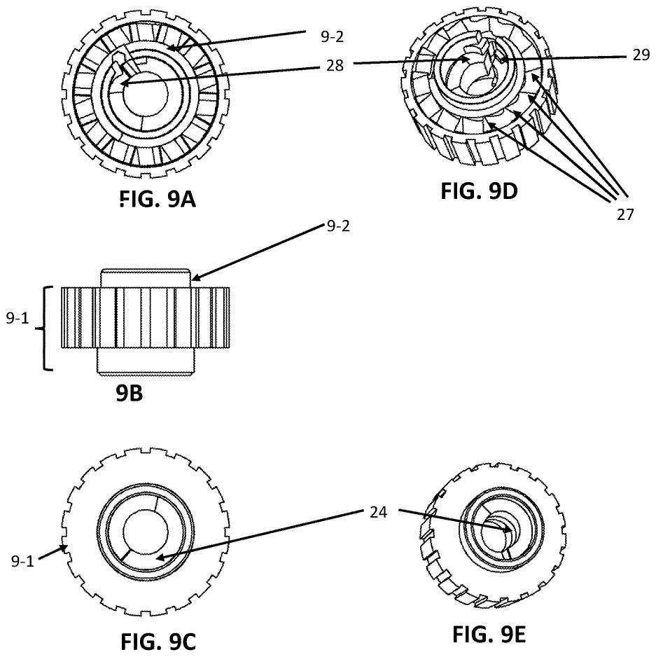

FIGS. 8A-8C illustrate a ratcheting feature of an exemplary syringe-based drug delivery device, such as the device of FIG. 3. The ratcheting feature may be used when advancing plunger rod 10-2 and drive rod 10-1 towards the start of dose position. FIG. 8A illustrates the device and the direction of rotation of dial 9-1. FIG. 8B illustrates an alternate view of the device in which the cover 6-1 is removed to expose ratchet 9-2 and beam 25, which is part of housing 6-2. The ratcheting design elements are magnified in FIG. 8C, showing flat surface 26 which may impact against flat surfaces on ratchet 9-2. In some embodiments, these flat surfaces may be edges 27, allowing rotation only in the direction shown by the arrow in FIG. 8A. Rotating the dial 9-1 and/or ratchet 9-2 may advance drive rod 10-1 during the setting of the drug dose. Therefore, retrograde movement of plunger rod 10-2 and drive rod 10-1 may be prevented by flat surface 26 and the flat surfaces represented by edge 27 (as shown in FIG. 9).

FIGS. 9A-E show different views of dial 9-1 and ratchet 9-2, which may be combined as one part in an exemplary syringe-based drug delivery device. The bottom view shows internal threads 24 which may guide protrusions 22 of drive rod 10-1 toward the position corresponding to a set dose. Once at the start of dose position, the surface of protrusion 22 most proximal to the user may rest on a flat surface 28. In some embodiments, the drive rod 10-1 may no longer be axially advanced, but the drive rod 10-2 (and hence, also plunger rod 10-2) may be prevented from retrograde travel by the flat surface 28. In some embodiments, the start of dose position may not be impacted by the decompression of plunger stopper 2. Another surface orthogonal to the surface of the protrusion 22 most proximal to the user may impact a flat surface (e.g., edge 29) when the user attempts to turn the dial 9-1 in the dose setting direction. This may prevent further rotation of the dial 9-1, which may indicate to the user that the start of dose position has been reached.

For those skilled in the art, it may be obvious that for certain applications (e.g., applications not requiring attenuation of force experienced by the user), the gear 7 may be not be included. In such embodiments, the plunger rod 10-2 and drive rod 10-1 may be one part (e.g., there may be just one plunger rod to which the plunger stopper is mounted or otherwise coupled). Excluding the teeth 18 and 19, one or more features of the plunger rod 10-2 and/or drive rod 10-1 may be consolidated as one rod.

In some embodiments, an external compressed gas source may be coupled to an exemplary syringe-based drug delivery device to provide the device with a high injection force required to deliver a sub-milliliter dose through a very fine injection needle (i.e., 37G or finer). In some embodiments, the delivery of the dose through such a device may or may not involve plunger stopper compression. The compressed gas source may be actuated using a foot-pedal, which may free the clinician's hands in case of injection into a sensitive area such as the subretinal space. In some embodiments, the device may enable slower injection speeds than would be possible manually. In order to facilitate transferring the drug from the vial into the syringe, priming the device, and enabling target volume delivery from a start of dose position to end of dose position, a user-selectable dial may be manipulated by the user. In some embodiments, the device may further comprise an adapter that connects the syringe-based module with the external compressed gas source. Unlike conventional syringe-based systems, this adapter may include dose reference markings to provide the indication of injection progress without the clinician having to loosen their grip on the syringe.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method comprising:
advancing a plunger rod of a syringe-based drug delivery device in an axial direction of the plunger rod from a start of dose position to an end of dose position to advance a plunger stopper within a barrel of the syringe-based drug delivery device for ejecting a dose of a fluid from a delivery conduit of the syringe-based drug delivery device, wherein the plunger stopper is compressed when the plunger rod first reaches the end of dose position, and a difference between the start of dose position and the end of dose position accounts for compression of the plunger stopper caused by resistance of the fluid to ejection through the delivery conduit.

2. The method of claim 1, wherein the end of dose position is defined by abutment between a portion of the plunger rod and a body of the syringe-based drug delivery device.

3. The method of claim 1, wherein the end of dose position is defined by at least one of a visual marker, an audible indicator, and a tactile indicator.

4. The method of claim 1, wherein the plunger stopper is mounted at one end to an end of the plunger rod such that the end of the plunger stopper that is mounted to the end of the plunger rod travels the difference between the start of dose position and the end of dose position.

5. The method of claim 1, wherein the plunger stopper is coupled to a drive rod that is at least partially disposed within the barrel of the syringe-based drug delivery device, and the drive rod and the plunger rod are operatively coupled such that the drive rod and the plunger rod translate different amounts.

6. The method of claim 1, wherein the plunger rod is axially locked at the end of dose position.

7. The method of claim 1, comprising, prior to advancing the plunger rod of the syringe-based drug delivery device from the start of dose position to the end of dose position, advancing the plunger rod to the start of dose position and waiting for the fluid to stop exiting the delivery conduit.

8. The method of claim 7, comprising visually, audibly, and/or tactilely indicating the start of dose position of the plunger rod.

9. The method of claim 1, wherein the plunger rod engages a ratchet configured to prevent the plunger rod from being pushed backward due to the compression of the plunger stopper.

10. The method of claim 1, comprising:
prior to advancing the plunger rod of the syringe-based drug delivery device from the start of dose position to the end of dose position, inserting the delivery conduit into an injection site; and
withdrawing the delivery conduit from the injection site prior to decompression of the plunger stopper.

11. The method of claim 10, wherein a difference between a start of dose position of a fluid contacting end of the plunger stopper and an end of dose position of the fluid contacting end of the plunger stopper is less than the difference between the start of dose position and the end of dose position of the plunger rod when the delivery conduit is withdrawn from the injection site.

12. A syringe-based drug delivery device comprising:
a housing comprising a barrel for containing a dose of a fluid;
a delivery conduit coupled to the barrel and configured for insertion into an injection site for delivering the dose of the fluid;
a plunger stopper positioned in the barrel;
a plunger rod operatively coupled to the plunger stopper to move the plunger stopper to deliver the dose of the fluid; and
at least one of a visual, audible, and tactile indicator of each of: (a) a start of dose position of the plunger rod and (b) an end of dose position of the plunger rod, wherein a difference between the start of dose position and the end of dose position accounts for compression of the plunger stopper during delivery of the dose of the fluid.

13. The syringe-based drug delivery device of claim 12, wherein the at least one of a visual, audible, and tactile indicator of the end dose position comprises an abutment between the plunger rod and a housing of the syringe-based drug delivery device that prevents the plunger rod from being further advanced.

14. The syringe-based drug delivery device of claim 12, wherein the plunger stopper is mounted at one end to an end of the plunger rod such that the end of the plunger stopper that is mounted to the end of the plunger rod travels the difference between the start of dose position and the end of dose position.

15. The syringe-based drug delivery device of claim 12, wherein the plunger stopper is mounted to a drive rod that is at least partially disposed within the barrel of the syringe-based drug delivery device, and the drive rod and plunger rod are operatively coupled such that the drive rod and the plunger rod translate different amounts.

16. The syringe-based drug delivery device of claim 12, wherein the plunger rod engages the housing at the end of dose position so that the plunger rod cannot move backward from the end of dose position.

17. The syringe-based drug delivery device of claim 12, comprising a ratchet configured to prevent the plunger rod from being pushed backward due to decompression of the stopper as the plunger is being advanced to the start of dose position.

18. The syringe-based drug delivery device of claim 12, wherein a difference between a start of dose position of a fluid contacting end of the plunger stopper and an end of dose position of the fluid contacting end of the plunger stopper is less than the difference between the start of dose position and the end of dose position of the plunger rod when the delivery conduit is withdrawn from the injection site.
